(19) United States
(12) Patent Application Publication
(10) Pub. No.: US 2021/0052848 A1
(43) Pub. Date: Feb. 25, 2021

(54) Reduced Trauma Micro-Preemie Catheter System

(71) Applicant: Timothy Alan Duvall, Reno, NV (US)

(72) Inventor: Timothy Alan Duvall, Reno, NV (US)

(21) Appl. No.: 16/548,460

(22) Filed: Aug. 22, 2019

(51) Int. Cl. A61M 25/00; A61M 25/06

(52) U.S. Cl. CPC A61M 25/0021; A61M 25/0662; A61M 5/3273; A61M 25/0097; A61M 25/0618

(57) ABSTRACT

A catheter system that includes a proximal hub having a body with a first end, a second end, and a first internal communication lumen. The catheter system includes an introducer apparatus connected to the proximal hub, the apparatus having a distal end and a second internal communication lumen in communication with the first internal communication lumen. A portion of the introducer apparatus extends from the second end of the proximal hub. The catheter system includes a distal hub having a body with a first end, a second end, and a third internal communication lumen in communication with the first internal communication lumen. The catheter system includes a catheter having a fourth internal communication lumen, the catheter extends through the first, second, and third internal communication lumens and a portion of the catheter extends from the distal end of the introducer apparatus. The introducer apparatus may be a non-splitting thin-walled sheath.

### FIELD OF THE DISCLOSURE

The embodiments described herein relate to apparatuses, systems, and methods for a reduced trauma micro-preemie catheter system.

### BACKGROUND

### Description of the Related Art

Peripherally inserted central catheters, midline catheters and peripheral intravenous catheters are commonly used to access the vascular system to deliver intravenous drugs and therapeutic agents. Generally, these catheters are introduced using a separate distinct medical introduction device, where the catheter is inserted through the device into the vessel and, which is then removed to allow for the placed vascular catheter to remain. The introducers need to have a large enough internal diameter (ID) to accommodate the diameter of the catheter. Additionally, the introducers need to have an adequate outside diameter (OD) in order to provide a mechanical stable introduction tube. Introduction tubes are generally split-walled tubes that allow the introduction tube to be split and removed leaving the catheter in place.

Such introducers many present problems when placing catheters in the vessels of very small patients such as micro preemies. A micro preemie is a baby who is born weighing less than 1 pound, 12 ounces (800 grams) or before 26 weeks gestation. One potential problem with these patients is that the target vessel ID maybe adequate to allow for the desired indwelling catheter ID, but that same vessel ID may not be adequate to accommodate the OD of the introducer device needed. Another problem is that the OD of the introducer may cause trauma due to the small ID of the vessels. Potential trauma may complicate such therapy, by compromising the patients' vessels as well as compromising the patents health. Additional disadvantages may exist.

### SUMMARY

The present disclosure is directed to apparatuses, systems, and methods for a reduced trauma micro-preemie catheter system.

An embodiment of the present disclosure is a catheter system comprising a proximal hub, the proximal hub comprising a body having a first end and a second end and a first internal communication lumen through the body. The catheter system includes an introducer apparatus having a distal end, the introducer apparatus connected to the proximal hub, the introducer apparatus having a second internal communication lumen in communication with the first internal communication lumen, wherein a first portion of the introducer apparatus is positioned within the first internal communication lumen and a second portion of the introducer apparatus extends from the second end of the proximal hub. The catheter system includes a distal hub, the distal hub comprising a body having a first end, a second end, and a third internal communication lumen through the body, wherein the third internal communication lumen is in communication with the first internal communication lumen. The catheter system includes a catheter having a fourth internal communication lumen, the catheter extends through the first, second, and third internal communication lumens and a portion of the catheter extends from the distal end of the introducer apparatus.

The introducer apparatus may be a non-splitting thin-walled sheath. The catheter may include a fifth internal communication lumen and the distal hub may include a sixth internal communication lumen. The distal end of the introducer apparatus may include a tipped end. The first end of the proximal hub may include a female slip luer mating portal and the second end of the distal hub may include a male slip mating assembly positioned within the female slip luer mating portion of the proximal hub. The first end of the distal hub may include a luer fitting. The proximal hub may include a pair of wings that extend from the body transverse to the first internal communication lumen. The catheter system may include a locking collar, wherein the locking collar secures the distal hub to the proximal hub.

An embodiment of the present disclosure is an apparatus comprising a proximal hub, the proximal hub comprising a body having a first end, a second end, and a first internal communication lumen through the body, wherein the first end is a female slip luer mating profile. The apparatus includes an introducer apparatus having a distal end, the introducer apparatus connected to the proximal hub, the introducer apparatus having a second internal communication lumen in communication with the first internal communication lumen, wherein a first portion of the introducer apparatus is positioned within the first internal communication lumen and a second portion of the introducer apparatus extends from the second end of the proximal hub, wherein the distal end is a tipped end and wherein the introducer apparatus is a non-splitting thin-walled sheath.

The first internal communication lumen and the second internal communication lumen may be positioned over a guidewire. A device may be passed through the first internal communication lumen and the second internal communication lumen to extend beyond the distal end of the introducer apparatus. The device may be a needle or a dilator assembly. Upon removal of the device from the first and second internal communication lumens, a catheter having a fourth internal communication lumen may be passed through the first and second internal communication lumens and a proximal hub may be connected to the first end of the distal hub, the proximal hub having a third internal communication lumen, wherein a portion of the catheter is positioned within the third internal communication lumen. The body of the proximal hub may comprise a pair of wings that extend from the body.

An embodiment of the present disclosure is a method comprising inserting a distal end of an introducer apparatus into a vessel, the introducer apparatus being connected to a proximal hub having a first internal communication lumen, the introducer apparatus having a second internal communication lumen in communication with the first internal communication lumen. The method includes inserting a catheter having a fourth internal communication lumen through the first and second internal communication lumens and into the vessel, a portion of the catheter being positioned within a third internal communication lumen of a distal hub. The method comprises connecting the proximal hub to the distal hub.

The method may include running the first and second internal communication lumens of the introducer apparatus and the proximal hub onto a guidewire positioned within the vessel prior to inserting the distal end of the introducer apparatus into the vessel, a portion of the guidewire being positioned within the vessel. The method may include removing the guidewire from the first and second internal communications lumens and the vessel prior to inserting the catheter. The method may include connecting a needle cannula that includes a needle to the proximal hub prior to inserting the distal end of the introducer apparatus into the vessel, the needle passes through the first and second internal communication lumens and a portion of the needle extends beyond the distal end of the introducer apparatus. The method may include removing the needle cannula prior to inserting the catheter.

The method may include connecting a dilator cannula that includes a dilator to the proximal hub prior to inserting the distal end of the introducer apparatus into the vessel, the dilator passes through the first and second internal communication lumens and a portion of the dilator extends beyond the distal end of the introducer apparatus. The method may include removing the dilator cannula prior to inserting the catheter. The method may include running the dilator, the first internal communication lumen, and second internal communication lumen onto a guidewire prior to inserting the distal end of the introducer apparatus into the vessel, a portion of the guidewire being positioned within the vessel. The method may include removing both the dilator cannula and the guidewire from the first and second internal communications lumens and the vessel prior to inserting the catheter.

### BRIEF DESCRIPTION OF THE DRAWINGS

FIG. 1 shows an embodiment of a proximal hub and introducer apparatus.

FIG. 2 shows the proximal hub and introducer apparatus of FIG. 1 positioned over a guidewire.

FIG. 3 shows a needle cannula connected to the proximal hub and introducer apparatus of FIG. 1 with a portion of the needle extending from a distal end of the introducer apparatus.

FIG. 4 shows a dilator cannula connected to the proximal hub and introducer apparatus of FIG. 1 with a portion of the dilator extending from a distal end of the introducer apparatus.

FIG. 5 shows an embodiment of a proximal hub and introducer apparatus.

FIG. 6 shows the proximal hub and introducer apparatus of FIG. 5 positioned over a guidewire.

FIG. 7 shows a needle cannula connected to the proximal hub and introducer apparatus of FIG. 5 with a portion of the needle extending from a distal end of the introducer apparatus.

FIG. 8 shows a dilator cannula connected to the proximal hub and introducer apparatus of FIG. 5 with a portion of the dilator extending from a distal end of the introducer apparatus.

FIG. 9 shows an embodiment of a distal hub and a catheter.

FIG. 10 shows an embodiment of a distal hub and a catheter.

FIG. 11 shows an embodiment of a catheter system.

FIG. 12 shows an embodiment of a catheter system.

FIG. 13 shows an embodiment of a catheter system.

FIG. 14 shows an embodiment of a catheter system.

FIG. 15 is a flow chart of an embodiment of a method of the present disclosure.

FIG. 16 is a cross-section view of an embodiment of a catheter having a single lumen.

FIG. 17 is a cross-section view of an embodiment of a catheter having dual lumen.

FIG. 18 shows an embodiment of a distal hub and a catheter.

While the disclosure is susceptible to various modifications and alternative forms, specific embodiments have been shown by way of example in the drawings and will be described in detail herein. However, it should be understood that the disclosure is not intended to be limited to the particular forms disclosed. Rather, the intention is to cover all modifications, equivalents and alternatives falling within the scope of the invention as defined by the appended claims.

### DETAILED DESCRIPTION

As discussed above, there is a need for a micro preemie catheter system that can both accommodate the OD of a catheter and reduce potential trauma to the patient's vessels. The introducer disclosed herein may be a thin-walled device, which used herein is a device having a wall thickness of 0.004 inches or less.

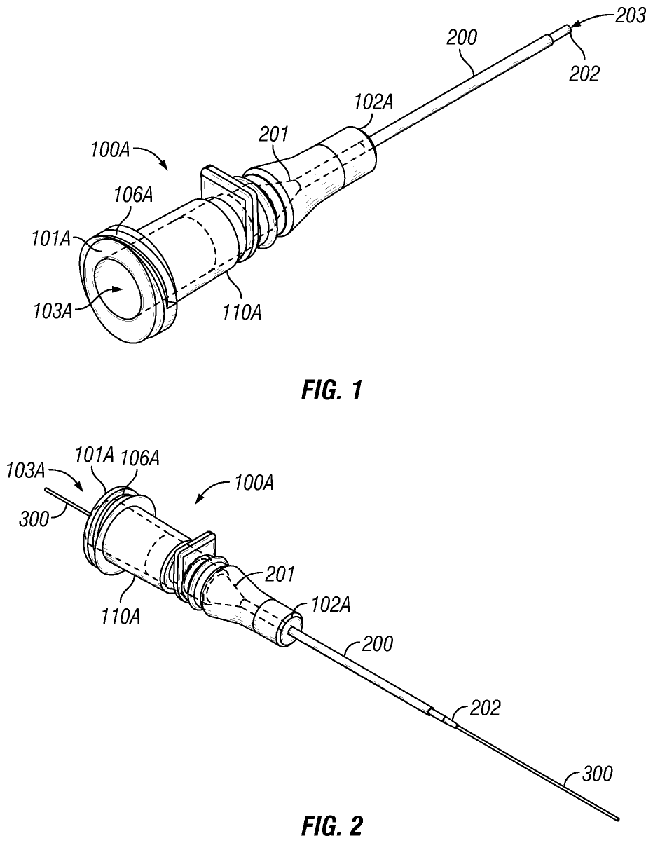

FIG. 1 shows an embodiment of a proximal hub 100A and introducer apparatus 200. The proximal hub 100A includes a body 110A having a first end 101A and a second end 102A with a first internal communication lumen 103A through the body 110A. The body 110A includes a flange 106A located at the first end 101A. The first end 101A of the proximal hub 100A may be a female slip luer mating portal as shown in FIG. 1.

The introducer apparatus 200 includes a first end, or proximal end, 201 located within the proximal hub 100A and a second end, or distal end, 202 that extends away from the proximal hub 100A. The second end 202 may be a tipped end as shown in FIG. 1. The introducer apparatus 200 includes a second internal communication lumen 203. The introducer apparatus 200 is a non-splitting thin-walled sheath. The introducer apparatus 200 may have a wall-thickness of 0.0035 inches. The wall-thickness may be varied on the application as would be appreciated by one of ordinary skill in the art. For example, the wall-thickness may be between 0.0005 inches and 0.004 inches.

The second internal communication lumen 203 of the introducer apparatus 200 is in communication with the first internal communication lumen 103A of the proximal hub 100A. The first and second internal communication lumens 103A, 203 of the proximal hub 100A and the connected introducer apparatus 200 may be used to insert a catheter into a vessel of a patient as discussed herein. FIG. 2 shows the assembly of the proximal hub 100A and the introducer apparatus 200 positioned on a guidewire 300. Although not shown, a portion of the guidewire 300 may be previously inserted into a vessel of a patient and may be used to guide the second end 202 introducer apparatus into the vessel as would be appreciated by one of ordinary skill in the art having the benefit of this disclosure. After positioning the distal end 202 of the introducer apparatus 200 into the vessel, the guidewire 300 may be withdrawn from the vessel enabling a catheter to be inserted into the vessel via the first and second internal communication lumens 103A, 203 of the proximal hub 100A and the connected introducer apparatus 200 as discussed herein.

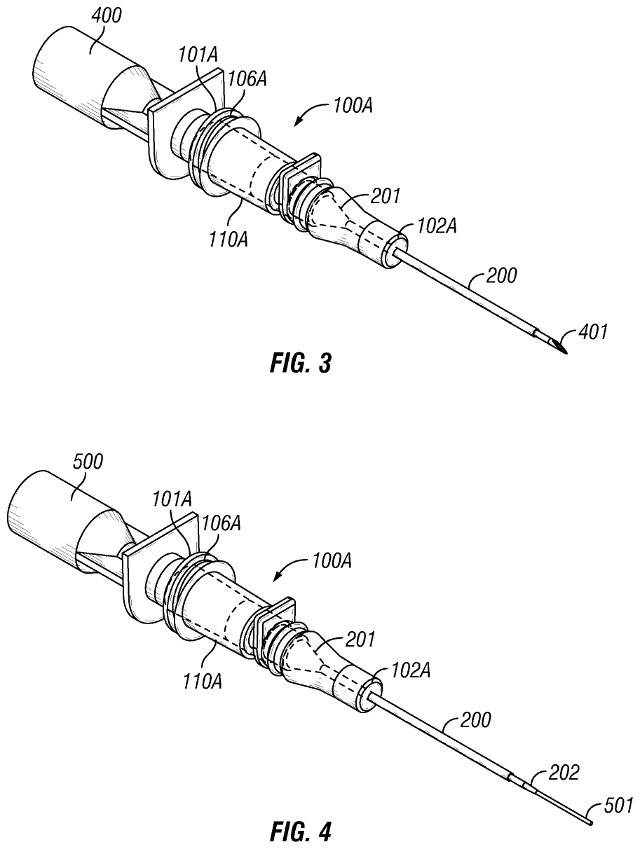

FIG. 3 shows a needle cannula 400 connected to the proximal hub 100A with a portion of the needle 401 of the needle cannula 400 extending from the distal end 202 of the introducer apparatus 200. The needle 401 may be used to pierce a vessel of a patient to insert both the needle 401 and distal end 202 of the introducer apparatus 200 into the vessel as would be appreciated by one of ordinary skill in the art having the benefit of this disclosure. After positioning the distal end 202 of the introducer apparatus 200 into the vessel, the needle cannula 400 may be disconnected from the proximal hub 100A to withdraw the needle 401 from the vessel enabling a catheter to be inserted into the vessel via the first and second internal communication lumens 103A, 203 of the proximal hub 100A and the connected introducer apparatus 200 as discussed herein.

FIG. 4 shows a dilator cannula 500 connected to the proximal hub 100A with a portion of the dilator 501 of the dilator cannula 500 extending from the distal end 202 of the introducer apparatus 200. The dilator 501 and the introducer apparatus 200 may be inserted into a vessel of a patient that may have been previously pierced with a needle. Alternatively, the dilator cannular 500, proximal hub 100A, and introducer apparatus 200 may be run onto a guidewire 300 with a portion of the guidewire 300 already positioned within a vessel of a patient. The guidewire 300 may be used to guide the dilator 501 and the distal end 202 of the introducer apparatus 200 into the vessel as would be appreciated by one of ordinary skill in the art having the benefit of this disclosure. After positioning the distal end 202 of the introducer apparatus 200 into the vessel, the dilator cannula 500 may be disconnected from the proximal hub 100A to withdraw the dilator 501 from the vessel. Further, the guidewire 300 may also be removed from the vessel to enable a catheter to be inserted into the vessel via the first and second internal communication lumens 103A, 203 of the proximal hub 100A and the connected introducer apparatus 200 as discussed herein.

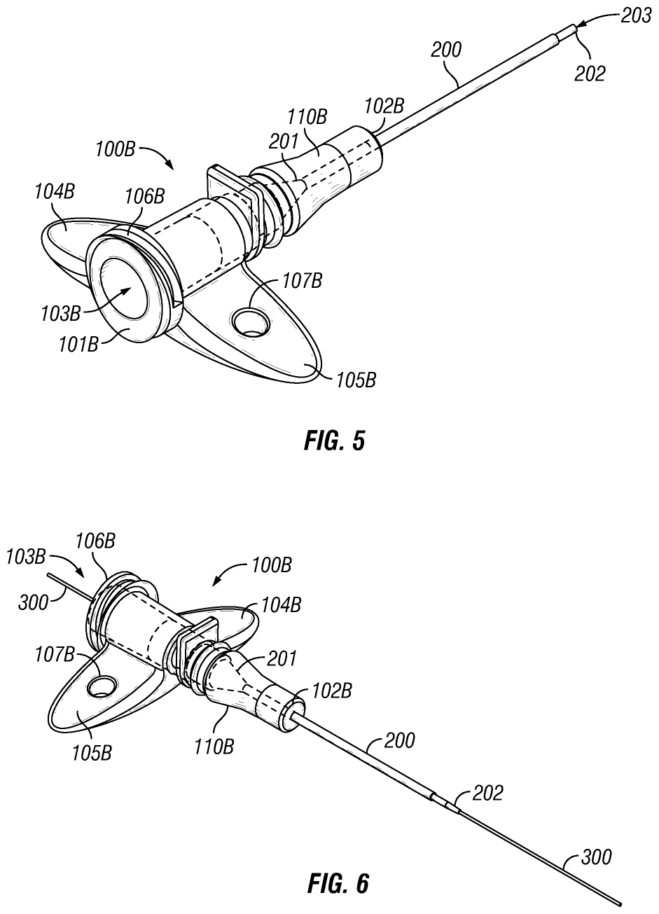

FIG. 5 shows an embodiment of a proximal hub 100B and introducer apparatus 200. The proximal hub 100B includes a body 110B having a first end 101B and a second end 102B with a first internal communication lumen 103B through the body 110B. The body 110B includes a flange 106B located at the first end 101B. The body 110B includes wings 104B, 105B that extend away from the body 110B. The wings 104B, 105B extend transverse to the first internal communication lumen 103B through the body 110B. The wings 104B, 105B may be used to secure the proximal hub 100B to a patient. The wings 104B, 105B may include one or more openings 107B that may be used to secure the proximal hub 100B to a patient. The first end 101A of the proximal hub 100A may be a female slip luer mating portal as shown in FIG. 5.

The introducer apparatus 200 includes a first end, or proximal end, 201 located within the proximal hub 100B and a second end, or distal end, 202 that extends away from the proximal hub 100B. The second end 202 may be a tipped end as shown in FIG. 5. The introducer apparatus 200 includes a second internal communication lumen 203. The introducer apparatus 200 is a non-splitting thin-walled sheath.

The second internal communication lumen 203 of the introducer apparatus 200 is in communication with the first internal communication lumen 103B of the proximal hub 100B. The first and second internal communication lumens 103B, 203 of the proximal hub 100B and the connected introducer apparatus 200 may be used to insert a catheter into a vessel of a patient as discussed herein. FIG. 6 shows the assembly of the proximal hub 100B and the introducer apparatus 200 positioned on a guidewire 300. Although not shown, a portion of the guidewire 300 may be previously inserted into a vessel of a patient and may be used to guide the second end 202 introducer apparatus into the vessel as would be appreciated by one of ordinary skill in the art having the benefit of this disclosure. After positioning the distal end 202 of the introducer apparatus 200 into the vessel, the guidewire 300 may be withdrawn from the vessel enabling a catheter to be inserted into the vessel via the first and second internal communication lumens 103B, 203 of the proximal hub 100B and the connected introducer apparatus 200 as discussed herein.

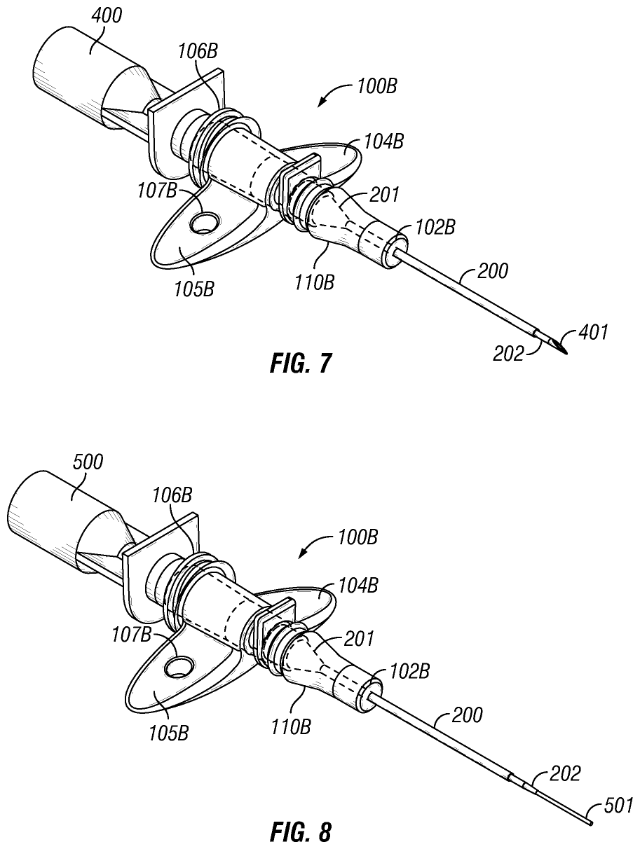

FIG. 7 shows a needle cannula 400 connected to the proximal hub 100B with a portion of the needle 401 of the needle cannula 400 extending from the distal end 202 of the introducer apparatus 200. The needle 401 may be used to pierce a vessel of a patient to insert both the needle 401 and distal end 202 of the introducer apparatus 200 into the vessel as would be appreciated by one of ordinary skill in the art having the benefit of this disclosure. After positioning the distal end 202 of the introducer apparatus 200 into the vessel, the needle cannula 400 may be disconnected from the proximal hub 100B to withdraw the needle 401 from the vessel enabling a catheter to be inserted into the vessel via the first and second internal communication lumens 103B, 203 of the proximal hub 100B and the connected introducer apparatus 200 as discussed herein.

FIG. 8 shows a dilator cannula 500 connected to the proximal hub 100B with a portion of the dilator 501 of the dilator cannula 500 extending from the distal end 202 of the introducer apparatus 200. The dilator 501 and the introducer apparatus 200 may be inserted into a vessel of a patient that may have been previously pierced with a needle. Alternatively, the dilator cannular 500, proximal hub 100B, and introducer apparatus 200 may be run onto a guidewire 300 with a portion of the guidewire 300 already positioned within a vessel of a patient. The guidewire 300 may be used to guide the dilator 501 and the distal end 202 of the introducer apparatus 200 into the vessel as would be appreciated by one of ordinary skill in the art having the benefit of this disclosure. After positioning the distal end 202 of the introducer apparatus 200 into the vessel, the dilator cannula 500 may be disconnected from the proximal hub 100B to withdraw the dilator 501 from the vessel. Further, the guidewire 300 may also be removed from the vessel to enable a catheter to be inserted into the vessel via the first and second internal communication lumens 103B, 203 of the proximal hub 100B and the connected introducer apparatus 200 as discussed herein.

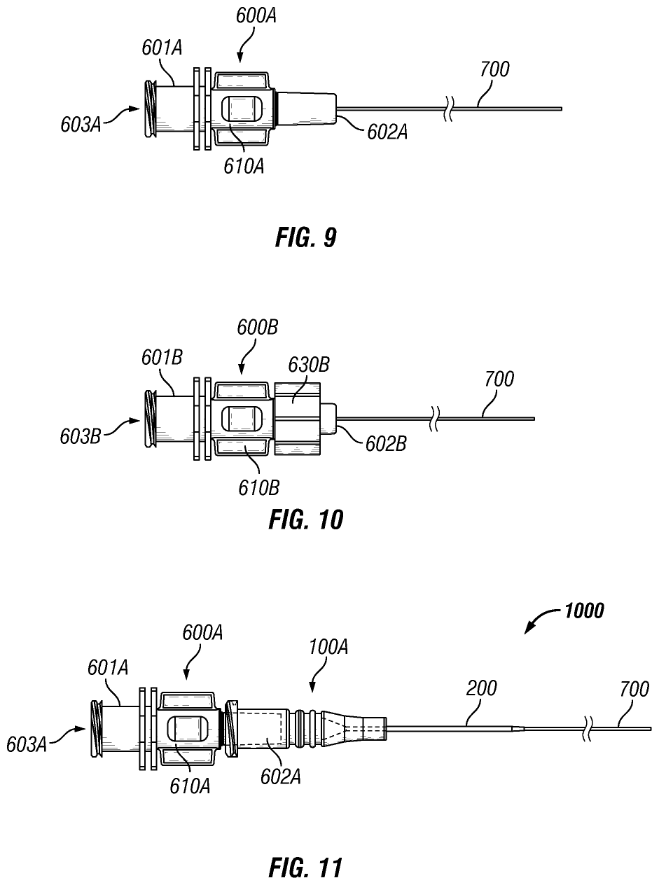

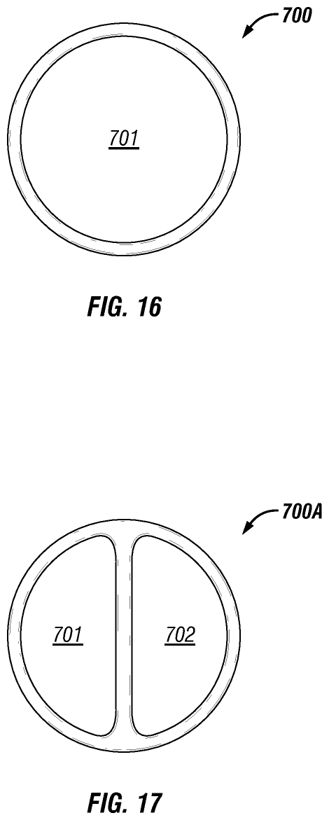

FIG. 9 shows an embodiment of a distal hub 600A and a catheter 700 positioned through the distal hub 600A. The distal hub 600A includes a body 610A having a first end 601A and a second end 602A with a third internal communication lumen 603A through the body 610A. The second end 602A of the distal hub 600A is a male slip mating assembly as shown in FIG. 9. The first end 601A of the distal hub 600A is a luer fitting as shown in FIG. 9. The catheter 700 is positioned through the third internal communication lumen 603A of the distal hub 600A. The catheter 700 has a fourth internal communication lumen 701 (shown in FIG. 16). The catheter 700 may include a fifth internal communication lumen 702 (shown in FIG. 17).

FIG. 10 shows an embodiment of a distal hub 600B and a catheter 700 positioned through the distal hub 600B. The distal hub 600B includes a body 610B having a first end 601B and a second end 602B with a third internal communication lumen 603B through the body 610B. The second end 602B of the distal hub 600B is a male slip mating assembly as shown in FIG. 10. The first end 601B of the distal hub 600B is a luer fitting as shown in FIG. 10. The distal hub 600B includes a lock nut 630B that may be used to secure the distal hub 600B to a proximal hub 100A, 100B as discussed herein. The catheter 700 is positioned through the third internal communication lumen 603B of the distal hub 600B. The catheter 700 has a fourth internal communication lumen 701 (shown in FIG. 16). The catheter 700 may include a fifth internal communication lumen 702 (shown in FIG. 17).

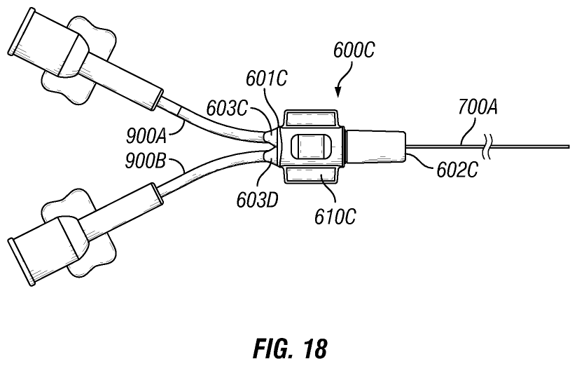

FIG. 18 shows an embodiment of a distal hub 600C and a catheter 700A positioned through the distal hub 600C. The catheter 700A is a dual lumen catheter best shown in FIG. 17. The two lumens of the dual lumen catheter 700A referred to herein as a fourth internal communication lumen and a fifth internal communication lumen. The distal hub 600C includes a body 610C having a first end 601C and a second end 602C with a two internal communication lumens 603C, 603D through the body 610C. The two internal communication lumens 603C, 603D referred to herein as a third internal communication lumen and a sixth internal communication lumen. The second end 602C of the distal hub 600C is a male slip mating assembly as shown in FIG. 17. The first end 601C of the distal hub 600C and dual internal communication lumens 603C, 603D are configured to receiving two input lines 900A, 900B. The dual lumen catheter 700A is positioned with the distal hub 600C and a first lumen 701 (shown in FIG. 17) is in communication with an internal lumen 603C through the distal hub 600C and the first input line 900A. A second lumen 702 (shown in FIG. 17) of the dual lumen catheter 700A is in communication with an internal lumen through the distal hub 600C and the second input line 900B. The distal hub 600C, input lines 900A, 900B and the catheter 700A may be used to provide two different treatments to a patient as would be appreciated by one of ordinary skill in the art having the benefit of this disclosure.

FIG. 11 shows an embodiment of a catheter system 1000. The catheter system 1000 includes a distal hub 600A connected to a proximal hub 100A with a catheter 700 extending through the distal hub 600A, proximal hub 100A, and an introducer apparatus 200 connected to the proximal hub 100A. The introducer apparatus 200 is a non-splitting thin-walled sheath that enables the catheter 700 to be inserted into a patient with minimal trauma to the patient, which may be a micro preemie. The proximal hub 100A is connected to the introducer apparatus 200 as discussed herein. The distal end 202 introducer apparatus 200 is first inserted into a vessel of a patient. As discussed herein, various methods may be used to insert the distal end 202 of the introducer apparatus 200 into the vessel. The catheter system 1000 could use the distal hub 600C of FIG. 18 and dual lumen catheter 700A of FIG. 17 in place of the distal hub 600A and catheter 700 as would be appreciated by one of ordinary skill in the art having the benefit of this disclosure.

For example, the proximal hub 100A and introducer apparatus 200 may be run onto a guidewire 300 with a portion of the guidewire 300 positioned within a vessel. The guidewire 300 may be used to guide the distal end 202 of the introducer apparatus 200 into the vessel. The guidewire 300 may then be removed and a catheter 700 positioned within the third internal communication lumen 603A of a distal hub 600A may be inserted through the first and second internal communication lumens 103A, 203 of the proximal hub 100A and the introducer apparatus 200. Once the end of catheter 700 is positioned at a desired location within the vessel, the proximal hub 100A and introducer apparatus 200 may be moved in a direction away from the vessel to mate up with the distal hub 600A. The proximal hub 100A and distal hub 600A are connected together to form the catheter system 1000 shown in FIG. 11. For example, the second end 602A of the distal hub 600A may be positioned within a portion of the proximal hub 100A. The second end 602A of the distal hub 600A may be male slip mating assembly positioned within a female slip luer mating portal of the first end 101A of the proximal hub 100A. The catheter system 1000 may then be secured to the patient and the patient may be treated with various therapy treatments through the catheter 700.

As another example, a needle cannula 400 may be connected to the proximal hub 100A and introducer apparatus 200 with a portion of the needle 401 extending from the distal end 202 of the introducer apparatus 200. The needle 401 may be used to insert the distal end 202 of the introducer apparatus 200 into a vessel of a patient. The needle cannula 400 may then be disconnected from the proximal hub 100A to withdraw the needle 401 from the vessel through the first and second internal communication lumens 103A, 203 of the proximal hub 100A and the introducer apparatus 200. A catheter 700 positioned within the third internal communication lumen 603A of a distal hub 600A may be inserted through the first and second internal communication lumens 103A, 203 of the proximal hub 100A and the introducer apparatus 200. Once the end of catheter 700 is positioned at a desired location within the vessel, the proximal hub 100A and introducer apparatus 200 may be moved in a direction away from the vessel to mate up with the distal hub 600A. The proximal hub 100A and distal hub 600A are connected together to form the catheter system 1000 shown in FIG. 11. The catheter system 1000 may then be secured to the patient and the patient may be treated with various therapy treatments through the catheter 700.

As another example, a dilator cannula 500 may be connected to the proximal hub 100A and introducer apparatus 200 with a portion of the dilator 501 extending from the distal end 202 of the introducer apparatus 200. The dilator 501 and the distal end 202 of the introducer apparatus 200 into a vessel of a patient either through a prior needle opening or onto a guidewire 300. The dilator cannula 500 may then be disconnected from the proximal hub 100A to withdraw the dilator 501 from the vessel through the first and second internal communication lumens 103A, 203 of the proximal hub 100A and the introducer apparatus 200. A catheter 700 positioned within the third internal communication lumen 603A of a distal hub 600A may be inserted through the first and second internal communication lumens 103A, 203 of the proximal hub 100A and the introducer apparatus 200. Once the end of catheter 700 is positioned at a desired location within the vessel, the proximal hub 100A and introducer apparatus 200 may be moved in a direction away from the vessel to mate up with the distal hub 600A. The proximal hub 100A and distal hub 600A are connected together to form the catheter system 1000 shown in FIG. 11. The catheter system 1000 may then be secured to the patient and the patient may be treated with various therapy treatments through the catheter 700.

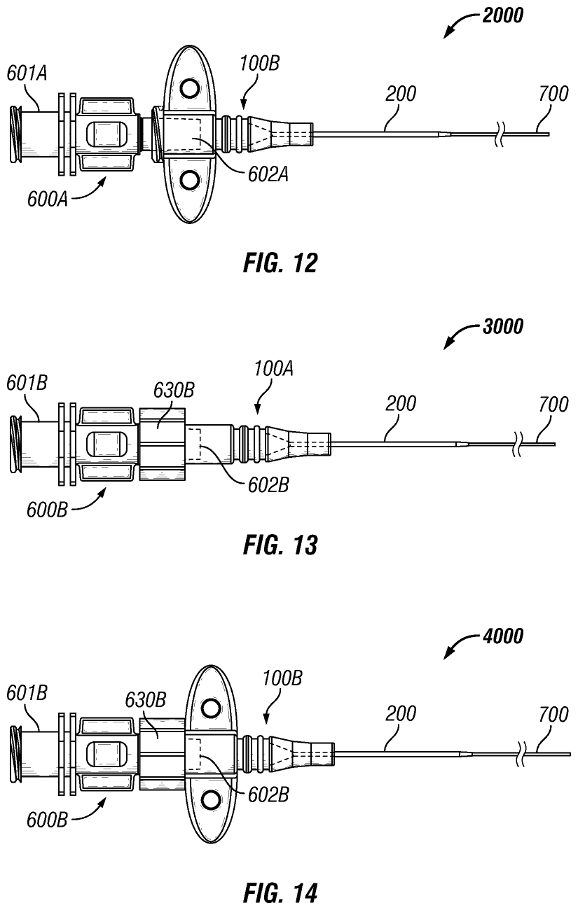

FIG. 12 shows an embodiment of a catheter system 2000. The catheter system 2000 includes a distal hub 600A connected to a proximal hub 100B with a catheter 700 extending through the distal hub 600A, proximal hub 100B, and an introducer apparatus 200 connected to the proximal hub 100B. The introducer apparatus 200 is a non-splitting thin-walled sheath that enables the catheter 700 to be inserted into a patient with minimal trauma to the patient, which may be a micro preemie. The proximal hub 100B is connected to the introducer apparatus 200 as discussed herein. The distal end 202 introducer apparatus 200 is first inserted into a vessel of a patient. The catheter system 2000 could use the distal hub 600C of FIG. 18 and dual lumen catheter 700A of FIG. 17 in place of the distal hub 600A and catheter 700 as would be appreciated by one of ordinary skill in the art having the benefit of this disclosure.

As discussed herein, various methods may be used to insert the distal end 202 of the introducer apparatus 200 into the vessel. For example, the distal end 202 of the introducer apparatus 200 may be inserted via a guidewire 300, a needle cannula 400, and/or a dilator cannula 500 as discussed herein. Once the distal end 202 of the introducer apparatus 200 is positioned within a vessel, the mechanism used to insert the distal end 202 of the introducer apparatus 200 may be removed as discussed herein enabling a catheter 700 to be inserted into the vessel via the first and second internal communication lumens 103B, 203 of the proximal hub 100B and the introducer apparatus 200. The proximal hub 100B may then be withdrawn to mate up with the distal hub 600A to form catheter system 2000. The catheter system 2000 may then be secured to the patient and the patient may be treated with various therapy treatments through the catheter 700. For example, the wings 104B, 105B of the proximal hub 100B may be used to secure the catheter system 2000 to the patient.

FIG. 13 shows an embodiment of a catheter system 3000. The catheter system 3000 includes a distal hub 600B connected to a proximal hub 100A with a catheter 700 extending through the distal hub 600B, proximal hub 100A, and an introducer apparatus 200 connected to the proximal hub 100A. The introducer apparatus 200 is a non-splitting thin-walled sheath that enables the catheter 700 to be inserted into a patient with minimal trauma to the patient, which may be a micro preemie. The proximal hub 100A is connected to the introducer apparatus 200 as discussed herein. The distal end 202 introducer apparatus 200 is first inserted into a vessel of a patient. The catheter system 3000 could use the distal hub 600C of FIG. 18 and dual lumen catheter 700A of FIG. 17 in place of the distal hub 600B and catheter 700 as would be appreciated by one of ordinary skill in the art having the benefit of this disclosure.

As discussed herein, various methods may be used to insert the distal end 202 of the introducer apparatus 200 into the vessel. For example, the distal end 202 of the introducer apparatus 200 may be inserted via a guidewire 300, a needle cannula 400, and/or a dilator cannula 500 as discussed herein. Once the distal end 202 of the introducer apparatus 200 is positioned within a vessel, the mechanism used to insert the distal end 202 of the introducer apparatus 200 may be removed as discussed herein enabling a catheter 700 to be inserted into the vessel via the first and second internal communication lumens 103A, 203 of the proximal hub 100A and the introducer apparatus 200. The proximal hub 100A may then be withdrawn to mate up with the distal hub 600B to form the catheter system 3000. The lock nut 630B of the distal hub 600B may be threaded onto flange 106A of the proximal hub 100A to secure the distal hub 600B to the proximal hub 100A. The catheter system 3000 may then be secured to the patient and the patient may be treated with various therapy treatments through the catheter 700.

FIG. 14 shows an embodiment of a catheter system 4000. The catheter system 4000 includes a distal hub 600B connected to a proximal hub 100B with a catheter 700 extending through the distal hub 600B, proximal hub 100B, and an introducer apparatus 200 connected to the proximal hub 100B. The introducer apparatus 200 is a non-splitting thin-walled sheath that enables the catheter 700 to be inserted into a patient with minimal trauma to the patient, which may be a micro preemie. The proximal hub 100B is connected to the introducer apparatus 200 as discussed herein. The distal end 202 introducer apparatus 200 is first inserted into a vessel of a patient. The catheter system 4000 could use the distal hub 600C of FIG. 18 and dual lumen catheter 700A of FIG. 17 in place of the distal hub 600B and catheter 700 as would be appreciated by one of ordinary skill in the art having the benefit of this disclosure.

As discussed herein, various methods may be used to insert the distal end 202 of the introducer apparatus 200 into the vessel. For example, the distal end 202 of the introducer apparatus 200 may be inserted via a guidewire 300, a needle cannula 400, and/or a dilator cannula 500 as discussed herein. Once the distal end 202 of the introducer apparatus 200 is positioned within a vessel, the mechanism used to insert the distal end 202 of the introducer apparatus 200 may be removed as discussed herein enabling a catheter 700 to be inserted into the vessel via the first and second internal communication lumens 103B, 203 of the proximal hub 100B and the introducer apparatus 200. The proximal hub 100B may then be withdrawn to mate up with the distal hub 600B to form the catheter system 4000. The lock nut 630B of the distal hub 600B may be threaded onto flange 106B of the proximal hub 100B to secure the distal hub 600B to the proximal hub 100B. The catheter system 4000 may then be secured to the patient and the patient may be treated with various therapy treatments through the catheter 700. For example, the wings 104B, 105B of the proximal hub 100B may be used to secure the catheter system 4000 to the patient.

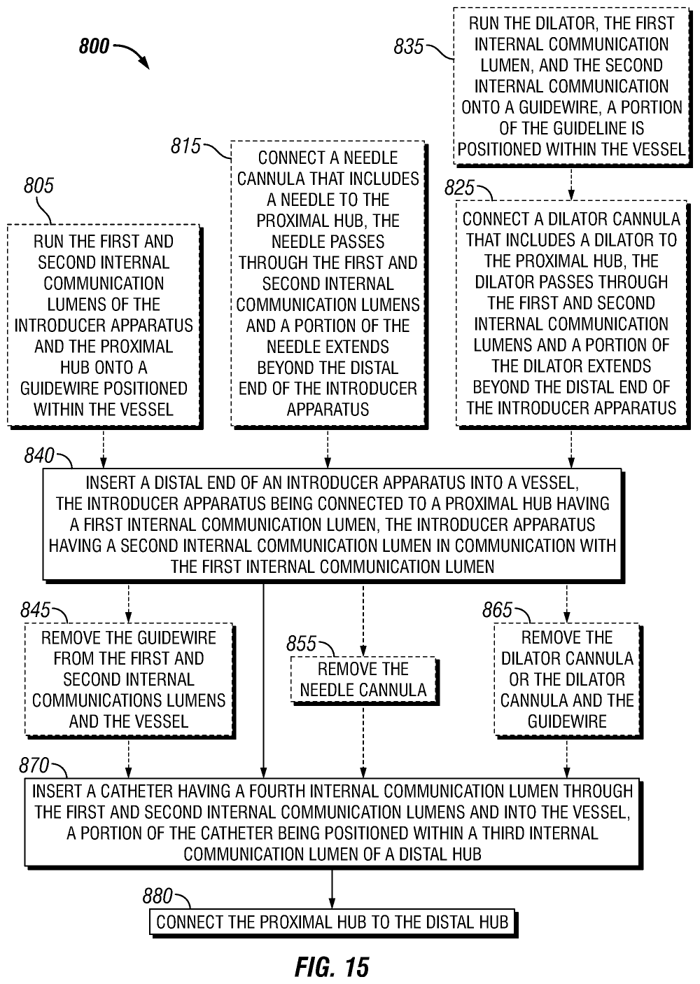

FIG. 15 is a flow chart of an embodiment of a method 800 of the present disclosure. The method 800 includes inserting a distal end of an introducer apparatus into a vessel, the introducer apparatus being connected to a proximal hub having a first internal communication lumen, the introducer apparatus having a second internal communication lumen in communication with the first internal communication lumen, at 840. The method 800 includes inserting a catheter having a fourth internal communication lumen through the first and second internal communication lumens and into the vessel, a portion of the catheter being positioned within a third internal communication lumen of a distal hub, at 870. The method 800 includes connecting the proximal hub to the distal hub, at 880.

Prior to inserting the distal end of the introducer apparatus into the vessel, the method 800 may include running the first and second internal communication lumens of the introducer apparatus and the proximal hub onto a guide wire positioned within the vessel, at 805. Prior to inserting the distal end of the introducer apparatus into the vessel, the method 800 may include connecting a needle cannula that includes a needle to the proximal hub, the needle passes through the first and second internal communication lumens and a portion of the needle extends beyond the distal end of the introducer apparatus, at 815. Prior to inserting the distal end of the introducer apparatus into the vessel, the method 800 may include connecting a dilator cannula that includes a dilator to the proximal hub, the dilator passes through the first and second internal communication lumens and a portion of the dilator extends beyond the distal end of the introducer apparatus, at 825. Prior to inserting the distal end of the introducer apparatus into the vessel, the method 800 may include running the dilator, the first internal communication lumen, and the second internal communication lumen onto a guidewire, a portion of the guidewire being positioned within the vessel, at 835.

Prior to inserting the catheter having the fourth communication lumen through the first and second internal communication lumens and into the vessel, the method 800 may include removing the guidewire from the first and second internal communications lumens and the vessel, at 845. Prior to inserting the catheter having the fourth communication lumen through the first and second internal communication lumens and into the vessel, the method 800 may include removing the needle cannula, at 855. Prior to inserting the catheter having the fourth communication lumen through the first and second internal communication lumens and into the vessel, the method 800 may include removing the dilator cannula or the dilator cannula and the guidewire, at 865.

FIG. 16 shows a cross-section of an embodiment of a catheter 700 having a single lumen, herein referred to as a fourth internal communication lumen 701. FIG. 17 shows a cross-section of an embodiment of a catheter 700A having dual lumen, herein referred to as a fourth internal communication lumen 701 and as a fifth internal communication lumen 702.

Although this disclosure has been described in terms of certain preferred embodiments, other embodiments that are apparent to those of ordinary skill in the art, including embodiments that do not provide all of the features and advantages set forth herein, are also within the scope of this disclosure. Accordingly, the scope of the present disclosure is defined only by reference to the appended claims and equivalents thereof.

What is claimed is:

1. A catheter system comprising:
a proximal hub, the proximal hub comprising a body having a first end and a second end and a first internal communication lumen through the body;
an introducer apparatus having a distal end, the introducer apparatus connected to the proximal hub, the introducer apparatus having a second internal communication lumen in communication with the first internal communication lumen, wherein a first portion of the introducer apparatus is positioned within the first internal communication lumen and a second portion of the introducer apparatus extends from the second end of the proximal hub;
a distal hub, the distal hub comprising a body having a first end, a second end, and a third internal communication lumen through the body, wherein the third internal communication lumen is in communication with the first internal communication lumen; and
a catheter having a fourth internal communication lumen, the catheter extends through the first, second, and third internal communication lumens and a portion of the catheter extends from the distal end of the introducer apparatus.

2. The catheter system of claim 1, wherein the introducer apparatus is a non-splitting thin-walled sheath.

3. The catheter system of claim 2, wherein the catheter includes a fifth internal communication lumen and the distal hub includes a sixth internal communication lumen.

4. The catheter system of claim 2, wherein the distal end of the introducer apparatus includes a tipped end.

5. The catheter system of claim 4, wherein the first end of the proximal hub comprises a female slip luer mating portal and the second end of the distal hub comprises a male slip mating assembly positioned within the female slip luer mating portion of the proximal hub.

6. The catheter system of claim 5, wherein the first end of the distal hub comprises a luer fitting.

7. The catheter system of claim 6, wherein the proximal hub includes a pair of wings that extend from the body transverse to the first internal communication lumen.

8. The catheter system of claim 7, further comprising a locking collar, wherein the locking collar secures the distal hub to the proximal hub.

9. The catheter system of claim 5, further comprising a locking collar, wherein the locking collar secures the distal hub to the proximal hub.

10. An apparatus comprising:
a proximal hub, the proximal hub comprising body having a first end, a second end, and a first internal communication lumen through the body, wherein the first end is a female slip luer mating profile; and
an introducer apparatus having a distal end, the introducer apparatus connected to the proximal hub, the introducer apparatus having a second internal communication lumen in communication with the first internal communication lumen, wherein a first portion of the introducer apparatus is positioned within the first internal communication lumen and a second portion of the introducer apparatus extends from the second end of the proximal hub, wherein the distal end is a tipped end and wherein the introducer apparatus is a non-splitting thin-walled sheath.

11. The apparatus of claim 10, wherein the first internal communication lumen and the second internal communication lumen may be positioned over a guidewire.

12. The apparatus of claim 10, further comprising a device passed through the first internal communication lumen and the second internal communication lumen to extend beyond the distal end of the introducer apparatus.

13. The apparatus of claim 12, wherein the device is a needle or a dilator.

14. The apparatus of claim 12, wherein upon removal of the device from the first and second internal communication lumens, a catheter having a fourth internal communication lumen may be passed through the first and second internal communication lumens and a proximal hub may be connected to the first end of the distal hub, the proximal hub having a third internal communication lumen, wherein a portion of the catheter is positioned within the third internal communication lumen.

15. The apparatus of claim 12, the body of the proximal hub comprises a pair of wings that extend from the body.

16. A method comprising:
inserting a distal end of an introducer apparatus into a vessel, the introducer apparatus being connected to a proximal hub having a first internal communication lumen, the introducer apparatus having a second internal communication lumen in communication with the first internal communication lumen;
inserting a catheter having a fourth internal communication lumen through the first and second internal communication lumens and into the vessel, a portion of the catheter being positioned within a third internal communication lumen of a distal hub; and
connecting the proximal hub to the distal hub.

17. The method of claim 16, further comprising:
running the first and second internal communication lumens of the introducer apparatus and the proximal hub onto a guidewire positioned within the vessel prior to inserting the distal end of the introducer apparatus into the vessel, a portion of the guidewire being positioned within the vessel; and
removing the guidewire from the first and second internal communications lumens and the vessel prior to inserting the catheter.

18. The method of claim 16, further comprising:
connecting a needle cannula that includes a needle to the proximal hub prior to inserting the distal end of the introducer apparatus into the vessel, the needle passes through the first and second internal communication lumens and a portion of the needle extends beyond the distal end of the introducer apparatus; and
removing the needle cannula prior to inserting the catheter.

19. The method of claim 16, further comprising:
connecting a dilator cannula that includes a dilator to the proximal hub prior to inserting the distal end of the introducer apparatus into the vessel, the dilator passes through the first and second internal communication lumens and a portion of the dilator extends beyond the distal end of the introducer apparatus; and
removing the dilator cannula prior to inserting the catheter.

20. The method of claim 19 comprising:
running the dilator, the first internal communication lumen, and second internal communication lumen onto a guidewire prior to inserting the distal end of the introducer apparatus into the vessel, a portion of the guidewire being positioned within the vessel; and
removing both the dilator cannula and the guidewire from the first and second internal communications lumens and the vessel prior to inserting the catheter.
